(19) United States
(12) Patent Application Publication
(10) Pub. No.: US 2006/0110341 A1
(43) Pub. Date: May 25, 2006

(54) Cosmetic compositions containing Sophora alopecuroides L. extracts

(75) Inventors: Bijan Harichian, Warren, NJ (US); Minliang Zou, Shanghai (CN); Hongqiang Wang, Shanghai (CN)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division Of Conopco, Inc.

(21) Appl. No.: 10/997,204

(22) Filed: Nov. 24, 2004

(51) Int. Cl. A61K 38/19; A61K 36/489; A61K 8/35

(52) U.S. Cl. 424/59; 424/62; 424/757

(57) ABSTRACT

Disclosed are extracts of Sophora alopecuroides L. as cosmetic skin lightening agents alone or in combination with other skin benefit agents and together with a cosmetic vehicle. Also disclosed are active ingredients in the extracts, including Alopecurone A, Alopecurone B, Sophoraflavone-G, Sophoraflavone-I, Sophoraflavone-K, and mixtures thereof. The inventive extracts, compositions and methods have effective skin lightening properties, may be easier to deliver to the skin and are available in nature.

### FIELD OF THE INVENTION

The invention relates to compositions for topical application to human skin which compositions contain extracts of Sophora Alopecuroides and to methods of using the compositions for lightening of skin.

### BACKGROUND OF THE INVENTION

Many people are concerned with the degree of pigmentation of their skin. For example, people with age spots or freckles may wish such pigmented spots to be less pronounced. Others may wish to reduce the skin darkening caused by exposure to sunlight or to lighten their natural skin color. To meet this need, many attempts have been made to develop products that reduce the pigment production in the melanocytes. However, the substances identified thus far tend to have either low efficacy or undesirable side effects, such as, for example, toxicity or skin irritation. Therefore, there is a continuing need for new skin lightening agents, with improved overall effectiveness. Moreover, many consumers are looking for natural ingredients for skin lightening application.

Mukenazu, JP7188245A relates to a compound contained in “Kukanzo” useful as an MRSA (methicillin-resistant Staphylococus aureus) antibacterial agent, antitumor active agent and anti-oral bacteria active agent. The compound is Alopecurone I and/or II, having chemical name of 4-hydroxy-2-(4-hydroxyphenyl)-3-(3,5-dihydroxyphenyl)-7-(2,4-dihydroxyphenyl)-9-(5-isopropenyl-1-methyl-hex-2-enyl)-5 H-2,3-dihydro(3,2-g) [1]-6,7-dihydrobenzopyran-5-one, or Alopecurone III. The compound is produced by extracting Kukanzo (root of Sophora alopecuroides, pulverized) with a solvent such as acetone, filtering the extract, concentrating the filtrate and separating and purifying by column chromatography.

Ku Gan Cao (Chinese pinyin) is a traditional medicine in China, which is the root of the plant Sophora alopecuroides L. This plant is a shrub, growing wild in fields, along river banks and in meadows, and widely available in Inner Mongolia, Xin Jiang autonomous region and Tibet in China, among other places. In traditional Chinese medicine, the cut pieces of the root are used as a fever reducer, as a pain reliever, and as an antibacterial agent. A different name, Ku Dou Geng, is also commonly used in the traditional Chinese medicine market.

Seven Alopecurones (alopecurone A-G) have been previously isolated from Sophora alopecuroides and identified by linuma, et al., “Six Flavonostilbenes and a Flavanone in Roots of Sophora Alopecuroides,” Phytochemistry, 38 (2): 519-525 (1995).

The present invention is based at least in part on the discovery that extracts of the plant Sophora alopecuroides L. and/or its active components, such as Alocpecurone A, Alopecurone B and newly identified components, have at least comparable and/or demonstrably better skin lightening activity than known skin lightening agents. The use of Sophora alopecuroides L. and/or its active components for cosmetic skin lightening applications has not heretofore been known.

### SUMMARY OF THE INVENTION

The present invention alleviates the deficiencies of the prior art and includes, in part, a novel composition for skin lightening containing cosmetically acceptable carrier and an organic solvent extract of Sophora alopecuroides L and method for skin lightening by applying the inventive composition. The inventive compositions contain about 0.000001 to about 50% of extract of Sophora alopecuroides, preferably, an organic solvent extract thereof. Preferably, the amount of the extract is about 0.00001% to about 10%, more preferably about 0.001% to about 7%, and even more preferably about 0.01% to about 5%, to attain optimum skin lightening activity at a minimum cost.

The present invention is based at least in part on the discovery that extracts of the plant Sophora alopecuroides have skin lightening activity. The concentrated extracts have an IC50 of about 12 micro-g/ml.

Alopecurones and Sophoraflavones were identified as active components of the extracts of Sophora alopecuroides L., having skin lightening activity. The present invention includes a composition and cosmetic method of skin lightening comprising applying to the skin a composition comprising an active ingredient selected from the group consisting of Alopecurone A, Alopecurone B, Sophoraflavone-G, Sophoraflavone-I, Sophoraflavone-K, and mixtures thereof.

In the preferred embodiment of the invention, further skin benefit agents may be included in the compositions and inventive method, such as alpha-hydroxy acids, beta-hydroxy acids, polyhydroxy acids, hydroquinone, t-butyl hydroquinone; Vitamin B and/or C derivatives; dioic acids; retinoids; resorcinol derivatives, particularly 4-substituted resorcinol derivatives; vanillic acid, betulinic acid, hydrolactin, and mixtures thereof. Organic and inorganic (e.g. micronized metal oxides) sunscreens may also be included. Organic sunscreens may include Benzophenone-3, Benzophenone-4, Benzophenone-8, Methoxycinnamate, Ethyl dihydroxypropyl-PABA, Glyceryl PABA, Homosalate, Methyl anthranilate, Octocrylene, Octyl dimethyl PABA, Octyl methoxycinnamate (PARSOL MCX), Octyl salicylate, PABA, 2-Phenylbenzimidazole-5-sulphonic acid, TEA salicylate, 3-(4-methylbenzylidene)-camphor, Benzophenone-1, Benzophenone-2, Benzophenone-6, Benzophenone-12, 4-Isopropyl dibenzoyl methane, Butyl methoxy dibenzoyl methane (PARSOL 1789), Etocrylene, and mixtures thereof.

The inventive compositions and methods have effective skin lightening properties, are cost-effective, and are available from natural sources.

### DETAILED DESCRIPTION OF THE INVENTION

The present invention relates to cosmetic compositions for lightening of human skin which compositions contain organic solvent extracts of the plant Sophora alopecuroides L. and to methods of using the compositions for lightening of skin. The present invention is based at least in part on the discovery that extracts of the plant Sophora alopecuroides have skin lightening activity. The concentrated extracts have an IC50 of about 12 micro-g/ml. Sophora alopecuroides L is the Latin name of an herb, a plant of the family Leguminosae, genus Sophora and species Alopecuroides. The shrub is 1-1.5 meters high. Its light green leaves, large and heart shaped, are 1.5-2.5 cm long and 0.7-1.0 cm wide. Its yellow flowers are bell shaped, about 8mm long. Seeds are commonly oval and light yellow.

Alopecurone was identified as one of the active components of the extracts of Sophora alopecuroides. Alopecurone A and B are described in more detail hereinbelow. Also identified as active components were novel molecules named herein Sophoraflavone I and Sophoraflavone K, described in more detail hereinbelow. Also identified was the skin lightening activity of Sophoraflavone G, also described in more detail hereinbelow.

The extracts of Sophora alopecuroides suitable for use in present compositions are organic solvent extracts, e.g., alcoholic extracts (methanol—MeOH, ethanol, isopropanol), ester such as ethyl acetate, or chloroform extracts. Ethanol is the preferred organic solvent because of its ability to extract the majority of the components of a wide variety of polarity.

The root of the herb is used to prepare the extract. The root is firstly ground to small pieces, or pulverized, to a particle size of an average diameter of about 0.1 millimeter to 10 millimeter and then immersed into extraction solvent, preferably 95% ethanol, at room temperature for about 2 to about 3 days while stirring occasionally. The extraction process is repeated one or two times. The extracts of the 2-3 times are combined, concentrated and dried at temperatures lower than about 60° C., to remove the solvent. The drying may be carried out under vacuum, for a period of about an hour. The resulting concentrated extract may be used as it is or after purification.

According to the present invention, extracts of Sophora alopecuroides L. are presented in the composition in an amount of about 0.000001% to about 50% by weight of the composition. Preferably, the amount of the extract is about 0.00001% to about 10%, more preferably about 0.001% to about 7%, and even more preferably about 0.01% to about 5%, to attain optimum skin lightening activity at a minimum cost.

As used herein, the term “cosmetic composition” is intended to describe compositions for topical application to human skin.

As used herein, the term “comprising” means including, made up of, composed of, consisting and/or consisting essentially of. Furthermore, in the ordinary meaning of “comprising,” the term is defined as not being exhaustive of the steps, components, ingredients, or features to which it refers.

The term “skin” as used herein includes the skin on the face, neck, chest, back, arms, axilla, hands, legs, and scalp.

Except in the examples, or where otherwise explicitly indicated, all numbers in this description indicating amounts of material or conditions of reaction, physical properties of materials and/or use are to be understood as modified by the word “about”. All amounts are by weight of the composition, unless otherwise specified.

It should be noted that in specifying any range of concentration, any particular upper concentration can be associated with any particular lower concentration.

Sophora alopecuroides L

The extracts according to the present invention are from a plant material of the Genus of Sophora; and Species of alopecuroides L., also known as Ku Gan Cao. Sophora alopecuroides L is a plant of the family Leguminosae. Sophora alopecuroides L. as used in accordance with the present invention, was harvested in the County of Ding Bian, Shanxi province, China. Sophora alopecuroides L. is then subjected to extraction.

Extraction Protocol

Dried roots are cut into small pieces and then soaked in 95% ethanol at room temperature for 2 or 3 days, followed by filtration to collect the filtrate. The same process is repeated for one more time. The two filtrates are combined and evaporated under vacuum at 50° C. to get the crude extract.

The crude extract is then dissolved in a solution of ethyl acetate, then partitioned with 5% sulphuric acid aqueous, to remove all the alkaloids in the crude extract. The upper layer of the solution is collected and concentrated under vacuum at 60° C. to get a powder.

The powder is then loaded onto a chromatographic column filled with a macro porous material Diaion HP-20. The column is firstly eluted with 40% of ethanol-water solution, and then eluted with 70% of ethanol-water solution. The eluate by 70% of ethanol-water solution is collected and concentrated under vacuum at 50° C. to get the final yellowish extract, which is proposed to use in formulation (hereinafter, concentrated extract). The 70% eluate is mostly concentrated in flavanoids to which the present invention is directed in one of its aspects.

Purification

The present invention is based on the discovery that Alopecurone A and Alopecurone B are showing tyrosinase inhibition activity. Two new molecules have been isolated in the work leading up to this invention named as Sophoraflavone I and Sophoraflavone K, respectively. The new molecules were isolated by loading the final extract from the extraction process into a silica gel column and eluted with chloroform-methanol system. Their structures were elucidated by a combination of Mass and NMR spectroscopy, as discussed hereinbelow.

### Optional Skin Benefit Agents

Preferred cosmetic compositions are those suitable for the application to human skin according to the method of the present invention, which optionally, but preferably, include a skin benefit agent in addition to the inventive extracts.

Suitable additional skin benefit agents include anti-aging, wrinkle-reducing, skin whitening, anti-acne, and sebum reduction agents. Examples of these include alpha-hydroxy acids, beta-hydroxy acids, polyhydroxy acids, hydroquinone, t-butyl hydroquinone, Vitamic B and C derivatives, dioic acids, retinoids; betulinic acid; vanillic acid; allantoin, a placenta extract; hydrolactin; and resorcinol derivatives.

### Cosmetically Acceptable Carrier

The cosmetically acceptable vehicle may act as a dilutant, dispersant or carrier for the skin benefit ingredients in the composition, so as to facilitate their distribution when the composition is applied to the skin.

The vehicle may be aqueous, anhydrous or an emulsion. Preferably, the compositions are aqueous or an emulsion, especially water-in-oil or oil-in-water emulsion, preferentially oil in water emulsion. Water when present will be in amounts which may range from 5 to 99%, preferably from 20 to 70%, optimally between 40 and 70% by weight.

Besides water, relatively volatile solvents may also serve as carriers within compositions of the present invention. Most preferred are monohydric C₁-C₃ alkanols. These include ethyl alcohol, methyl alcohol and isopropyl alcohol. The amount of monohydric alkanol may range from 1 to 70%, preferably from 10 to 50%, optimally between 15 to 40% by weight.

Emollient materials may also serve as cosmetically acceptable carriers. These may be in the form of silicone oils and synthetic esters. Amounts of the emollients may range anywhere from 0.1 to 50%, preferably between 1 and 20% by weight.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from 3 to 9, preferably from 4 to 5, silicon atoms. Linear volatile silicone materials generally have viscosities less than about 5 centistokes at 25° C. while cyclic materials typically have viscosities of less than about 10 centistokes. Nonvolatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 25 million centistokes at 25° C. Among the preferred non-volatile emollients useful in the present compositions are the polydimethyl siloxanes having viscosities from about 10 to about 400 centistokes at 25° C.

Among the ester emollients are:
(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, myristyl myristate, stearyl stearate and arachidyl behenate.
(5) Sterol esters, of which cholesterol fatty acid esters are examples.

Fatty acids having from 10 to 30 carbon atoms may also be included as cosmetically acceptable carriers for compositions of this invention. Illustrative of this category are pelargonic, lauric, myristic, palmitic, stearic, isostearic, hydroxystearic, oleic, linoleic, ricinoleic, arachidic, behenic and erucic acids.

Humectants of the polyhydric alcohol-type may also be employed as cosmetically acceptable carriers in compositions of this invention. The humectant aids in increasing the effectiveness of the emollient, reduces scaling, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycol or sodium hyaluronate. The amount of humectant may range anywhere from 0.5 to 30%, preferably between 1 and 15% by weight of the composition.

Thickeners may also be utilized as part of the cosmetically acceptable carrier of compositions according to the present invention. Typical thickeners include crosslinked acrylates (e.g. Carbopol 982), hydrophobically-modified acrylates (e.g. Carbopol 1382), taurate polymer, cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0001 to 5%, usually from 0.001 to 1%, optimally from 0.01 to 0.5% by weight.

Collectively the water, solvents, silicones, esters, fatty acids, humectants and/or thickeners will constitute the cosmetically acceptable carrier in amounts from 1 to 99.9%, preferably from 80 to 99% by weight.

An oil or oily material may be present, together with an emulsifier to provide either a water-in-oil emulsion or an oil-in-water emulsion, depending largely on the average hydrophilic-lipophilic balance (HLB) of the emulsifier employed.

Surfactants may also be present in cosmetic compositions of the present invention. Total concentration of the surfactant will range from 0.1 to 40%, preferably from 1 to 20%, optimally from 1 to 5% by weight of the composition. The surfactant may be selected from the group consisting of anionic, nonionic, cationic and amphoteric actives. Particularly preferred nonionic surfactants are those with a C₁₀-C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di- fatty acid esters of ethylene glycol; fatty acid monoglyceride; sorbitan, mono- and di- C₈-C₂₀ fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C₈-C₂₀ acyl isethionates, acyl glutamates, C₈-C₂₀ alkyl ether phosphates and combinations thereof.

### Optional Components

In the cosmetic compositions of the invention, there may be optionally added plasticizers; calamine; antioxidants; chelating agents; as well as sunscreens.

Other adjunct minor components may also be incorporated into the cosmetic compositions. These ingredients may include coloring agents, pigments, opacifiers, and perfumes. Amounts of these other adjunct minor components may range anywhere from 0.001% up to 20% by weight of the composition.

Sunscreens. For use as sunscreen, metal oxides may be used alone or in mixture and/or in combination with organic sunscreens. Examples of organic sunscreens include but are not limited those set forth in the table below.

The amount of the organic sunscreens in the cosmetic composition is preferably in the range of about 0.1 wt % to about 10 wt %, more preferably about 1 wt % to 5 wt %. Preferred organic sunscreens are PARSOL MCX and Parsol 1789, due to their effectiveness and commercial availability.

TABLE 1

| CTFA Name | Trade Name | Supplier |
|---|---|---|
| Benzophenone-1 | UVINUL 400 | BASF Chemical Co. |
| Benzophenone-2 | UVINUL D-50 | BASF Chemical Co. |
| Benzophenone-3 | UVINUL M-40 | BASF Chemical Co. |
| Benzophenone-4 | UVINUL MS-40 | BASF Chemical Co. |
| Benzophenone-6 | UVINUL D-49 | BASF Chemical Co. |
| Benzophenone-8 | SPECRA-SORB UV-24 | American Cyanamide |
| Benzophenone-12 | UVINUL 408 | BASF Chemical Co. |
| Methoxycinnamate | BERNEL HYDRO | Bernel Chemical |
| Ethyl dihydroxypropyl-PABA | AMERSCREEN P | Amerchol Corp. |
| Glyceryl PABA | NIPA G.M.P.A. | Nipa Labs. |
| Homosalate | KEMESTER HMS | Hunko Chemical |
| Methyl anthranilate | SUNAROME UVA | Felton Worldwide |
| Octocrylene | UVINUL N-539 | BASF Chemical Co. |
| Octyl dimethyl PABA | AMERSCOL | Amerchol Corp. |
| Octyl methoxycinnamate | PARSOL MCX | Bernel Chemical |
| Octyl salicylate | SUNAROME WMO | Felton Worldwide |
| PABA | PABA | National Starch |
| 2-Phenylbenzimidazole-5-sulphonic acid | EUSOLEX 232 | EM Industries |
| TEA salicylate | SUNAROME W | Felton Worldwide |
| 3-(4-methylbenzylidene)-camphor | EUSOLEX 6300 | EM Industries |
| 4-Isopropyl dibenzoyl methane | EUSOLEX 8020 | EM Industries |
| Butyl methoxy dibenzoyl methane | PARSOL 1789 | Givaudan Corp. |
| Etocrylene | UVINUL N-35 | BASF Chemical Co. |

### Use of the Composition

The herbal extracts, compositions and methods according to the invention are intended primarily as a personal care product for topical application to human skin to lighten the skin, to reduce the degree of pigmentation in the skin, or to even the skin tone.

In use, a small quantity of the composition, for example from 1 to 5 ml, is applied to exposed areas of the skin, from a suitable container or applicator and, if necessary, it is then spread over and/or rubbed into the skin using the hand or fingers or a suitable device.

### Product Form and Packaging

The cosmetic composition of the invention can be formulated as a lotion having a viscosity of from 4,000 to 10,000 mPas, a fluid cream having a viscosity of from 10,000 to 20, 000 mPas, or a cream having a viscosity of from 20,000 to 100,000 mPas or above. The composition can be packaged in a suitable container to suit its viscosity and intended use by the consumer. For example, a lotion or fluid cream can be packaged in a bottle or a roll-ball applicator or a propellant-driven aerosol device or a container fitted with a pump suitable for finger operation. When the composition is a cream, it can simply be stored in a non-deformable bottle or squeeze container, such as a tube or a lidded jar. When the composition is a solid or semi-solid stick, it may be packaged in a suitable container for manually or mechanically pushing out or extruding the composition.

The invention accordingly also provides a closed container containing a cosmetically acceptable composition as herein defined.

The following examples are by way of example, not by way of limitation, of the principles of the present invention, to illustrate the best mode of carrying out the invention.

### EXAMPLE 1

This example illustrates in vitro tyrosinase inhibition activity of the inventive plant extracts of Sophora alopecuroides L.

Original root freshly collected in the County of Ding Bian, ShanXi province, China, obtained via a local agency, Xin Zheng, which deals in raw materials of Traditional Chinese Medicine, was used.

The extracts of Sophora alopecuroides L. used throughout the examples that follow were prepared in accordance with the procedures set forth hereinabove, i.e., concentrated extracts.

Method

MelanoDerm Tissue Model Method

A MelanoDerm tissue equivalent model (MatTek MEL-300) containing melanocytes obtained from dark skin individuals was utilized. MelanoDerms were cultured as per the supplier instructions.

Medium was replaced every two days over a 14-day period and treatments including the natural extracts were added to the medium phase at a final concentration of 50 and 100-micrograms per ml. Positive controls were included, i.e., kojic acid and hydroquinone, which are known skin lightening agents. During each medium change, a portion of the waste was analyzed for levels of lactate dehydrogenase as an indicator of cellular toxicity and stress. At no time was the level of lactate dehydrogenase above the vehicle control solvents (water or ethanol).

Determination of melanin content within each MelanoDerm was performed by absorbance measurement at 475 nm. IC50 was determined by Tyrosinase inhibition assay. The assay uses 0.5 mM tyrosine in sodium phosphate buffer (pH 7.00) as substrate and the mushroom tyrosinase (Sigma) level is 60.5 U/mL. The reaction rate in the first 6.5 min is calculated based on the absorbance change at 475 nm.

TABLE 2A
MelanoDerm Skin Equivalent Model Results (Extraction solvent:
95% ethanol) (Melanoderm Results tested under 50 micro-g/ml)

| | IC50* | % Melanin to | Visual |
|---|---|---|---|
| Latin name | (micro-g/ml) | Control (Melanoderm) | Toxicity |
| Sophora | 12 micro-g/ml | 80.77% | N |
| alopecuroides L. | | | |
| (concentrated | | | |
| extract) | | | |

*IC50 refers to the concentration of the extract required to inhibit 50% of the tyrosinase activity.

TABLE 2B
Tyrosinase Inhibition Activity: Components

| | | | Concentration(% wt) |
|---|---|---|---|
| | | | (In concentrated |
| Formula | Compound | IC50 (μM) | extract) |
| 3 | Alopecurone A | 9 | 31.1 |
| 2 | Alopecurone B | 5 | 14.6 |
| 4 | Sophoraflavone G | 14 | 12.9 |
| 1 | Sophoraflavone I | 15 | 4.1 |
| 5 | Sophoraflavone K | 1 | 4.1 |
| Total | — | — | 66.8 |
| — | Kojic acid | 46.2 | |
| | | (6 micro-g/ml) | |
| — | Hydroquinone | 36.4 | |
| | | (4 micro-q/ml) | |

### EXAMPLE 2

Concentrated extract of Sophora Alopecuroides L. was analyzed by Mass and NMR spectroscopy to identify the following active compounds, as detailed below:
1: Sophoraflavone I
2: Alopecurone B
3: Alopecurone A
4: Sophoraflavone G
5: Sophoraflavone K
X Terra TM RP-18, 5 um, 4.6×150 mm Column, wave length 280 nm Gradient CH3CN in water from 30% to 100% in 45 minutes

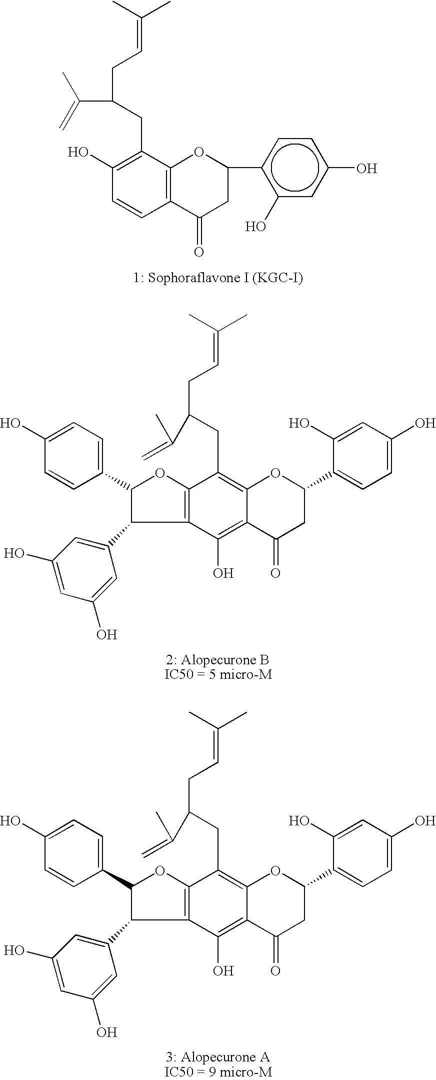

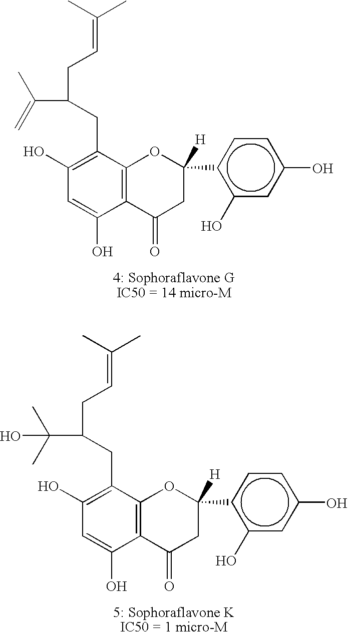

### EXAMPLE 3

Cosmetic compositions within the scope of the invention were prepared.

A base formulation shown in the Table below was made by heating phase A ingredients to 70 to 85° C. with stirring. Phase B ingredients were heated in a separate container to 70 to 85° C. with stirring. Then, phase A was added into phase B while both phases were kept at 70 to 85° C. The mixture was stirred for at least 15 minutes at 70 to 85° C., then cooled.

TABLE 3

| | a | b | |
|---|---|---|---|
| Inqredients | % wt. | % wt. | Phase |
| Isostearyl Palmitate | 6.00 | 6.00 | A |
| C12-C15 Alkyl Octanoate | 3.00 | 3.00 | A |
| PEG-100 Stearate | 2.00 | 2.00 | A |
| Glyceryl Hydroxystearate | 1.50 | 1.50 | A |
| Stearyl Alcohol | 1.50 | 1.50 | A |
| Stearic acid | 3.00 | 4.00 | A |
| TEA, 99% | 1.20 | 1.20 | B |
| Dimethicone | 1.00 | 1.00 | A |
| Sorbitan Monostearate | 1.00 | 1.00 | A |
| Magnesium Aluminum Silicate | 0.60 | 0.60 | B |
| Vitamin E acetate | 0.10 | 0.10 | A |
| Cholesterol | 0.50 | 0.50 | A |
| Simethicone | 0.01 | 0.01 | B |
| Xanthan gum | 0.20 | 0.20 | B |
| Hydroxyethylcellulose | 0.50 | 0.50 | B |
| Propylparaben | 0.10 | 0.10 | B |
| Disodium EDTA | 0.05 | 0.05 | B |
| Butylated hydroxytoluene | 0.05 | 0.05 | B |
| Sophora alopecuroides L. extract | 0.05 | 2.00 | B |
| Niacinamide | 1.00 | 1.00 | B |
| Metal oxide | 2.50 | 5.00 | B |
| Methylparaben | 0.15 | 0.15 | B |
| Water | BAL* | BAL* | B |
| Total | 100.00 | 100.00 | B |

*BAL means Balance.

### EXAMPLE4

Additional cosmetic compositions within the scope of the invention were prepared as follows:
1. Heat Phase A to 80° C.
2. Heat Phase B to 75° C. in a separate container
3. Add B to A and mix with heat off for 30 min.
4. At 50° C. add Phase C and mix for 10 min.

The concentrated extract of Sophora alopecuroides L. of Example 1 was used.

TABLE 4

| | Wt % | Wt % | Wt % | |
|---|---|---|---|---|
| Component | A | B | C | Phase |
| water, DI | BALANCE | BALANCE | BALANCE | A |
| disodium EDTA | 0.05 | 0.05 | 0.05 | A |
| magnesium aluminum | 0.6 | 0.6 | 0.6 | A |
| silicate | | | | |
| methyl paraben | 0.15 | 0.15 | 0.15 | A |
| Simethicone | 0.01 | 0.01 | 0.01 | A |
| butylene glycol 1,3 | 3.0 | 3.0 | 3.0 | A |
| Hydroxyethylcellulose | 0.5 | 0.5 | 0.5 | A |
| glycerine, USP | 2.0 | 2.0 | 2.0 | A |
| xanthan gum | 0.2 | 0.2 | 0.2 | A |
| Triethanolamine | 1.2 | 1.2 | 1.2 | B |
| stearic acid | 3.0 | 3.0 | 3.0 | B |
| propyl paraben NF | 0.1 | 0.1 | 0.1 | B |
| glyceryl | 1.5 | 1.5 | 1.5 | B |
| hydroxystearate | | | | |
| stearyl alcohol | 1.5 | 1.5 | 1.5 | B |
| isostearyl palmitate | 6.0 | 6.0 | 6.0 | B |
| C12-15 alcohols | 3.0 | 3.0 | 3.0 | B |
| octanoate | | | | |
| Dimethicone | 1.0 | 1.0 | 1.0 | B |
| cholesterol NF | 0.5 | 0.5 | 0.5 | B |
| sorbitan stearate | 1.0 | 1.0 | 1.0 | B |
| Micronized titanium | 5.0 | 5.0 | 5.0 | C |
| dioxide | | | | |
| tocopheryl acetate | 0.1 | 0.1 | 0.1 | B |
| PEG-100 stearate | 2.0 | 2.0 | 2.0 | B |
| sodium stearoyl | 0.5 | 0.5 | 0.5 | B |
| lactylate | | | | |
| hydroxycaprylic acid | 0.1 | 0.1 | 0.1 | C |
| Sophora alopecuroides | 10.0 | — | — | C |
| L. extract | | | | |
| Sophoraflavone I | — | 5.0 | — | C |
| Sophoraflavone K | — | — | 12.0 | C |
| PARSOL MCX | 2.4 | 2.4 | 2.4 | C |
| alpha-bisabolol | 0.2 | 0.2 | 0.2 | C |

### EXAMPLES 5-12

A set of additional compositions were prepared within the scope of the present invention and are listed in the table below.

The concentrated extract of Sophora alopecuroides L. of Example 1 was used.

TABLE 5

| | | Examples (wt. %) | | | | | | | |
|---|---|---|---|---|---|---|---|---|---|
| | | 5 | | | | | | | |
| | | acid soap | | | | | | | |
| Ingredients | Phase | base | 6 | 7 | 8 | 9 | 10 | 11 | 12 |
| Stearic acid | A | 17.9 | 17.9 | 17.9 | 17.9 | 17.9 | 17.9 | 17.9 | 17.9 |
| Sodium cetearyl | A | | 2.2 | | 1 | 1.5 | 2 | 3 | 2 |
| sulfate* (emulsifier) | | | | | | | | | |
| Myrj 59* (emulsifier) | A | | | 2 | 2 | 2 | 2 | 2 | 1 |
| Span 60* (emulsifiers) | A | | | 2 | 2 | 2 | 2 | 2 | 1 |
| Sophora alopecuroides | B | 0.05 | 0.05 | 2.0 | 2.0 | 3.5 | 3.5 | 5.0 | 10.0 |
| L. extract | | | | | | | | | |
| Micronized Zinc Oxide | B | 2.50 | 5.00 | 5.00 | 2.50 | 2.50 | 5.00 | 2.50 | 5.00 |
| KOH, 22% (form in situ | | 2.20 | | | | | | | |
| soap with stearic acid) | | | | | | | | | |
| Octyl methoxycinnamate | | 2.50 | | | 2.50 | 2.50 | | 2.50 | |
| Water | B | BAL | BAL | BAL | BAL | BAL | BAL | BAL | BAL |
| Glycerin | B | 1 | 1 | 1 | 1 | 1 | 1 | 1 | 1 |

It should be understood that the specific forms of the invention herein illustrated and described are intended to be representative only. Changes, including but not limited to those suggested in this specification, may be made in the illustrated embodiments without departing from the clear teachings of the disclosure. Accordingly, reference should be made to the following appended claims in determining the full scope of the invention.

What is claimed is:

1. A cosmetic method of skin lightening comprising applying to the skin a composition comprising:
a. about 0.000001 wt % to about 50 wt % of a Sophora alopecuroides extract; and
b. a cosmetically acceptable carrier.

2. The method of claim 1, wherein said composition further comprises a sunscreen.

3. The method of claim 2, wherein said sunscreen is a micronized metal oxide.

4. The method of claim 1, wherein said extract comprises about 0.00001% to about 10% of said composition.

5. The method of claim 1, wherein said extract comprises about 0.001% to about 7% of said composition.

6. The method of claim 1, wherein said extract comprises about 0.01% to about 5% of said composition.

7. The method of claim 1, wherein said extract has an IC50 of about 12 micro-g/ml.

8. The cosmetic method of claim 1, wherein said Sophora alopecuroides L. extract is an organic solvent extract.

9. The cosmetic method according to claim 1, wherein said composition further comprises a skin benefit agent selected from the group consisting of alpha-hydroxy acids, beta-hydroxy acids, polyhydroxy acids, hydroquinone, t-butyl hydroquinone, Vitamin B and/or C derivatives, dioic acid, retinoids, resorcinol derivatives, vanillic acid, betulinic acid, hydrolactin, and mixtures thereof.

10. The cosmetic method of claim 1, wherein said composition further comprises an organic sunscreen selected from the group consisting of Benzophenone-1, Benzophenone-2, Benzophenone-3, Benzophenone-4, Benzophenone-8, Methoxycinnamate, Ethyl dihydroxypropyl-PABA, Glyceryl PABA, Homosalate, Methyl anthranilate, Octocrylene, Octyl dimethyl PABA, Octyl methoxycinnamate (PARSOL MCX), Octyl salicylate, PABA, 2-Phenylbenzimidazole-5-sulphonic acid, TEA salicylate, 3-(4-methylbenzylidene)-camphor, Benzophenone-6, Benzophenone-12, 4-Isopropyl dibenzoyl methane, Butyl methoxy dibenzoyl methane (PARSOL 1789), Etocrylene, and mixtures thereof.

11. A cosmetic composition for skin lightening comprising:
a. about 0.000001 to about 50% of an organic solvent extract Sophora alopecuroides L.; and
a. a cosmetically acceptable carrier.

12. The cosmetic composition of claim 11, wherein said extract comprises about 0.00001% to about 10% of said composition.

13. The cosmetic composition of claim 11, wherein said extract comprises about 0.001% to about 7% of said composition.

14. The cosmetic composition of claim 11, wherein said extract comprises an active ingredient selected from the group consisting of Alopecurone A, Alopecurone B, Sophoraflavone-G, Sophoraflavone-I, Sophoraflavone-K, and mixtures thereof.

15. The cosmetic composition of claim 11, wherein said composition further comprises a skin benefit agent selected from the group consisting of alpha-hydroxy acids, beta-hydroxy acids, polyhydroxy acids, hydroquinone, t-butyl hydroquinone, Vitamin B and C derivatives, dioic acids, retinoids, resorcinol derivatives, vanillic acid, betulinic acid, hydrolactin, and mixtures thereof.

16. The cosmetic composition of claim 11, wherein said composition further comprises an organic sunscreen selected from the group consisting of Benzophenone-1, Benzophenone-2, Benzophenone-3, Benzophenone-4, Benzophenone-6, Benzophenone-8, Methoxycinnamate, Ethyl dihydroxypropyl-PABA, Glyceryl PABA, Homosalate, Methyl anthranilate, Octocrylene, Octyl dimethyl PABA, Octyl methoxycinnamate (PARSOL MCX), Octyl salicylate, PABA, 2-Phenylbenzimidazole-5-sulphonic acid, TEA salicylate, 3-(4-methylbenzylidene)-camphor, Benzophenone-12, 4-Isopropyl dibenzoyl methane, Butyl methoxy dibenzoyl methane (PARSOL 1789), Etocrylene, and mixtures thereof.

17. A cosmetic composition comprising an active ingredient selected from the group consisting of Alopecurone A, Alopecurone B, Sophoraflavone-G, Sophoraflavone-I, Sophoraflavone-K, and mixtures thereof.

18. The cosmetic composition of claim 17, wherein said composition comprises an active ingredient Alopecurone A or B.

19. A cosmetic method of skin lightening comprising applying to the skin the composition of claim 17.

20. The cosmetic method of skin lightening of claim 19, wherein said composition comprises an active ingredient selected from the group consisting of Sophoraflavone-G, Sophoraflavone-I, Sophoraflavone-K, and mixtures thereof.
